(19) United States
(12) Patent Application Publication
(10) Pub. No.: US 2001/0005502 A1
(43) Pub. Date: Jun. 28, 2001

(54) Monofluoro phosphorylated macromolecules

(76) Inventors: Alison Foster, Bebington (GB); Christopher David Gibbs, Bebington (GB); Anne Hilton, Bebington (GB); Ezat Khoshdel, Bebington (GB); Steven Rannard, Bebington (GB)

Correspondence Address: UNILEVER, PATENT DEPARTMENT, 45 RIVER ROAD, EDGEWATER, NJ, 07020, US

(21) Appl. No.: 09/737,684

(22) Filed: Dec. 15, 2000

(30) Foreign Application Priority Data
Dec. 17, 1999 (EP) 99310184.9

(51) Int. Cl. A61K 7/16; C08F 4/00; C08F 251/00; C08F 253/00; C08F 255/00; C08F 257/00; C08F 259/00

(52) U.S. Cl. 424/57; 525/255

(57) ABSTRACT

A stable oral composition comprising a natural or synthetic macromolecule with a monofluorophosphate moiety covalently bonded thereto.

The present invention relates to an oral composition comprising a macromolecule with a monofluorphosphate group grafted thereto.

The anti-caries effect of fluoride is well documented and monofluorophosphate is a well-known source of fluoride in oral care compositions. The sodium salt is used in many of the oral care compositions on the market today.

Unfortunately, excessive fluoride has toxic side-effects, commonly known as fluorosis, and the levels of fluoride permitted in oral care compositions is restricted for this reason. Added to the fact that it is difficult to deliver a substance within the oral cavity due to salivation, and particularly during rinsing when brushing the teeth, it is not surprising that there is much prior art relating to increasing the delivery of fluoride.

The prior art includes many disclosures relating to improved, longer lasting fluoridation including the suggestion that a regular release of small aliquots of fluoride at a high frequency is more cariostatic than fewer doses of higher concentrations (Regolati, Helv. Odont. Acta., Suppl. IX, 1975, pp 95-130).

There are also many disclosures relating to new molecules capable of providing an improvement of fluoride delivery. For example, WO 92/12983 (Allied-Signal) discloses a method of fluorinating using N-fluoro pyridinium pyridine heptafluoro diborate and U.S. Pat. No. 4,105,759 (Schreiber) describes the use of novel bis long chain (C8-18) amine monofluorophosphates in caries prophylaxis. The monofluorophosphates described therein are salts of the bis amine.

U.S. Pat. No. 4,020,019 (Soldati) discloses anti-caries agents in the form of films which are produced upon the interaction between certain novel polyethylenimine mono- and difluorophosphates of various molecular weights with tooth surfaces.

However, all of the compounds disclosed in Soldati are mono- and dimonofluorophosphate salts of the polyethylenimines and this means that they cannot be formulated in typical oral care compositions since the salt will not be stable in the presence of ingredients typically present in oral care compositions, particularly anionic surfactants. The only oral care composition exemplified in the disclosure is a mouthwash comprising glycerine, ethanol and water in addition to polyethylenimine difluorophosphate.

U.S. Pat. No. 3,997,504 (Plymale) discloses a polymerisable organic phosphoryl monofluoride. However, the active polymer is used for filling cavities in the tooth and it is designed to be made available at the site of action by polymerising in situ. Further, the material is to be applied by a dental practitioner and not by the consumer.

There are also many instances of fluoride or a fluoride source being covalently bonded to a molecule, which behaves as a slow-release fluoride source

U.S. Pat. No. 4,011,310 (Carter Wallace) describes novel fluorophosphate salts of alkylamines. The compounds are made by combining aqueous or organic solutions of linear or branched alkylamines with aqueous or organic solutions of mono- or difluorophosphoric acids.

Such a process limits the size of the molecule as a similar method for monofluorophosphorylating a macromolecule or a molecule with a plurality of monofluorophosphate binding would be much more difficult due to its three-dimensional configuration, particularly when a high monofluorophosphate content is required, and also because of the presence of other monofluorophosphate groups already bonded which provide steric hindrance to the addition of further similar groups.

Despite the prior art, there remains a need for materials which increase the delivery of fluoride in the oral cavity. There also remains a need for the use of such agents for every day use by the regular consumer.

Accordingly, in a first aspect, the present invention provides a stable oral composition which comprises a natural or synthetic macromolecule which has a monofluorophosphate moiety covalently bonded thereto.

The term macromolecule is meant generically and includes any molecule of molecular weight greater than 500.

In a preferred embodiment the macromolecule is a polymer, by which is meant a large molecule built up from smaller sub-units or monomers.

It is an essential feature of the invention that the monofluorophosphate moiety is covalently bonded to the macromolecule.

The monofluorophosphate is covalently conjugated to the macromolecule by way of a suitable binding site, e.g. a carboxylic acid group, an amine group, an alcohol group, a phosphate or a suitable leaving group. Such suitable leaving groups include but are not limited to halides (iodide, bromide, chloride), tosylate, brosylate, nosylate, mesylate, betylate, alkyl fluorosulfonate, alkyl perchlorate, triflate, nonaflate and tresylate. Preferable binding sites include a carboxylic acid group, an amine group, a phosphate group or a suitable leaving group. The most preferred binding group is an amine group which provides a X-N-P moiety which is easier to manufacture and may provide better fluoride release during use.

Preferably the macromolecule comprises at least two and preferably more monofluorophosphate moiety binding sites.

In a second aspect the invention provides a method of making a macromolecule with a monofluorophosphate moiety covalently bonded thereto.

A natural or synthetic macromolecule with a monofluorophosphate moiety covalently bonded thereto can be made by monofluorophosphorylating the macromolecule directly or by monofluorophosphorylating the monomer and polymerising said functionalised monomer.

The monomer or polymer may be functionalised with monofluorophosphate by any method common in the art. Examples of processes for functionalisation of monomer or macromolecule can be understood by reference to the following three methods found in the prior art:

reaction of a phosphate with 2,-4 dinitrobenzene as described in Percival M D, et al J. Org. Chem. (1992) 57, 811; and Wittman R (1963) 96, p771;

reaction of an alcohol with monofluorophosphoric acid as described in Parente J E, et al J. Am. Chem. Soc. (1984) 106, p8156;

substitution of a leaving group with sodium monofluorophosphate;

(i) reaction of an alcohol with SMFP via the alkyl triflate as described in Ambrose M G, et al J. Org. Chem. (1983) 48, p674.

(ii) reaction of a halide with sodium monofluorophosphate as described in Saunders B C, et al J. Chem. Soc. (1948) p695.

The functionalisation of a monomer or macromolecule with monofluorophosphate can also be carried out by reacting an alcohol with phosphorus oxychloride and then reacting the resulting phosphoryl chloride with sodium fluoride or triethylamine trihydrofluoride to form the phosphoryl fluoride. This can then be hydrolysed using sodium hydroxide solution to form the covalently conjugated monofluorophosphate.

It is to be understood that the reaction conditions in the cited examples can be routinely modified by the man skilled in the art to effect improved yield of product and the substrates used will influence such modifications where necessary.

Polymerisation of the functionalised monomer may be done by any of the methods common in the art, e.g. free-radical polymerisation; anionic polymerisation; cationic polymerisation; or step-growth polymerisation. An advantage of step-growth polymerisation is that the desired binding sites for the monofluorophosphate can be targeted without the polymerisation sites being affected. Two examples of step-growth polymerisation are ring-opening polymerisation and transesterification.

The oral composition according to the invention is a stable oral composition. By stable is meant that it can be formulated so that it may be used in a conventional oral care product such as a toothpaste for example. This is in contrast with a product where the bulk of the macromolecule with monofluorophosphate conjugated thereto is made in situ.

It also means that the product is capable of being used on a regular basis by a regular consumer instead of being applied by a dental practitioner who is trained in the art of applying the product so that in situ polymerisation may occur according to a complicated routine.

The composition according to the invention may be any oral, non-food composition, e.g. toothpaste and may be in the form of a gel, paste, gum or any other suitable type. Typically the oral composition may comprise from 0.001 to 10% by weight of the macromolecule according to claim 1. Preferably the oral composition will comprise from 0.01 to 5% by weight and most preferably from 0.1 to 3% by weight of the macromolecule according to claim 1.

The composition according to the invention may also preferably comprise a foaming agent. Preferred foaming agents include surfactants, particularly anionic surfactants such as the alkali-metal alkyl sulphates. The most common example is sodium lauryl sulphate (SLS).

Typical foaming agents are present in an amount which is capable of providing effective foaming of the product during use. Most consumers associate foaming with cleaning, i.e. if the product foams during use then it must be cleaning as well.

Typical amounts of foaming agents such as SLS range from 0.1 to 3.5% by weight of the total composition, preferably from 0.5 to 3% by weight and especially from 1 to 2.5% by weight.

The composition according to the invention may also comprise ingredients which are common in dentifrices. Examples of such ingredients include: antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium-, calcium-, magnesium- and stannous fluoride, aminefluorides, disodium monofluorophosphate, sodium trimeta phosphate and casein;

plaque buffers such as urea, calcium lactate, calcium glycerophosphate and strontium polyacrylates;

vitamins such as Vitamin C;

plant extracts;

desensitising agents, e.g. potassium citrate, potassium chloride, potassium tartrate, potassium bicarbonate, potassium oxalate, potassium nitrate and strontium salts;

anti-calculus agents, e.g. hypophosphite-containing polymers, organic phosphonates and phosphocitrates etc.;

gum protection agents, e.g. vegetable oils such as sunflower oil, rape seed oil, soybean oil and safflower oil; silicone oil; and hydrocarbon oil. The gum protection agent may be an agent capable of improving the permeability barrier of the gums. A complete description of agents capable of improving the permeability barrier of the gum is found in our copending application PCT/EP99/03368;

biomolecules, e.g. bacteriocins, antibodies, enzymes, etc.;

flavours, e.g. peppermint and spearmint oils;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials;

humectants such as glycerol, sorbitol, propyleneglycol, xylitol, lactitol etc.;

binders and thickeners such as sodium carboxymethylcellulose, xanthan gum, gum arabic etc. as well as synthetic polymers such as polyacrylates and carboxyvinyl polymers such as Carbopol®;

buffers and salts; and

other optional ingredients that may be included are e.g. bleaching agents such as peroxy compounds e.g. potassium peroxydiphosphate, effervescing systems such as sodium bicarbonate/citric acid systems, colour change systems, and so on.

In a further aspect the invention provides for the use of a composition according to the invention as an anti-caries composition.

In yet a further aspect the invention provides for the use of a macromolecule with a monofluorophosphate group conjugated thereto in the manufacture of a medicament for the remineralisation of teeth.

The invention will now be described in more detail by way of the following examples:

### EXAMPLE 1

The following reaction serves to illustrate the polymerisation of functionalised monomer.

A mixture of monofluorophosphate conjugated hydroxyethyl methacrylate (5.00 g, 0.02 mol), and water (5 ml) was stirred and purged with nitrogen for 30 minutes at room temperature.

To this was then added a solution of potassium persulfate (5 mg) in water (1 ml) and the resulting mixture was purged with nitrogen for a further 5 minutes. The reaction mixture was then heated at 65° C. for 6 hours. The polymer which had formed was cooled and filtered off using suction filtration with a fine filter paper, washed with cold acetone, dried in air, then dried in a vacuum desiccator over P₂O₅.

The presence of the monofluorophosphorylated macromolecule was analysed using standard NMR techniques. ¹H (500 MHz), ¹⁹F (470 MHz) and ³¹P (202 MHz) NMR spectra were recorded on a Bruker DRX500 spectrometer. ¹H NMR used tetramethylsilane as an internal standard, ¹⁹F NMR used trichlorofluoromethane as an external standard and ³¹P NMR used phosphoric acid as an external standard.

¹H NMR (500 MHz, D₂O) δ_{H} 0.8-1.1 (broad m); 1.9-2.0 (broad m) and 4.0-4.3 (broad m)

³¹ P NMR (202 MHz, D₂O) δ_{P} -4.6 (d, ¹J_{PF}=931.2 Hz)

¹⁹F NMR (470 MHz, D₂O) δ_{F} -80.9 (d, ¹J_{FP}=931.1 Hz)

### EXAMPLE 2

The following reaction serves to illustrate the functionalisation of a phosphate covalently conjugated to a monomer, polymer or other macromolecule and consists essentially of a reaction of a phosphate with 2,4-dinitrofluorobenzene modified from a procedure by Percival, M. D.; Witerhs, S. G.; J. Org. Chem., 1992, 57, 811 and Wittman, R.; Chem. Ber., 1963, 96, 771.

The phosphate (sodium salt) (3.00 g, 10.00 mmol) on the macromolecule or monomer was converted to the acid form by passing an aqueous solution through a DOWEX 50 X8 ion exchange resin (H⁺ form) into triethylamine (2.78 ml, 20.00 mmol). The aqueous solution was reduced in vacuo to leave the triethylammonium salt. The salt was dissolved in acetonitrile (15 ml) and to this was added triethylamine (0.42 ml, 3.00 mmol) and 2,4-dinitrofluorobenzene (1.57 ml, 12.5 mmol). The resulting mixture was stirred at room temperature for 24 hours in a flask protected with a calcium chloride guard tube. The solvent was removed in vacuo and water (50 ml) was added to the residue. The water layer was extracted with diethyl ether (2×50 ml). To the aqueous phase was added acidic Amberlite IR-120 until the solution became colourless. The resin and the precipitate were filtered off by passing through celite and washed with cold water. The filtrate was extracted with ether (4×30 ml) and the aqueous phase was collected and neutralised with sodium carbonate solution. The water was removed in vacuo to yield the fully functionalised product.

### EXAMPLE 3

The following reaction illustrates the functionalisation of either a monomer, polymer or other macromolecule and involves the reaction of an alcohol group on said monomer, polymer, macromolecule with monofluorophosphoric acid modified from a procedure by Parente, J. E.; Risley, J. M.; Van Etten, R. L.; J. Am. Chem. Soc,; 1984, 106, 8156.

Triethylamine (1.21 g, 12.00 mmol) was added to a stirred solution of the alcohol (30.00 mmol) and monofluorcphosphoric acid (0.59 g, 6.00 mmol). Tricholoroacetonitrile (4.33 g, 30.00 mmol) was then added and, after the exotherm had subsided, the reaction mixture was stirred at room temperature for 4 hours. The solution was cooled and the excess trichloroacetonitrile was removed in vacuo. Water (27 ml) was added and the solution extracted with diethyl ether (3×20 ml). Cyclohexylamine (5.95 g, 60.00 mmol) was added to the aqueous extract and the solution was cooled to 0° C. whereupon acetone (88 ml, 1.20 mol) was added. The solution was allowed to crystallise overnight at 4° C. to precipitate the product.

The cyclohexylamine salt was converted to the sodium salt by passing down a DOWEX 50 ×8 column (Na⁺ form).

### EXAMPLE 4

The following reaction illustrates how a macromolecule may be functionalised with monofluorophosphate. It involves the fluorination of a phosphoric acid function.

The phosphorylated macromolecule (2.38 mmol) was dissolved in dry dichloromethane (10 ml) and to this was added a fluorinating agent, e.g. Deoxofluor ([bis(2-methoxyethyl)amino]sulphur trifluoride) (0.58 g, 2.62 mmol). The reaction mixture was stirred at room temperature for 4 days under nitrogen and then poured into ice/water (10 ml). The reaction mixture was neutralised with NaHCO₃ solution, the two layers were separated and the aqueous extract was extracted with dichloromethane (2×20 ml). The aqueous extract was then reduced in vacuo to leave the monofluorphosphorylated macromolecule product.

### EXAMPLE 5

This example illustrates how reaction of an alcohol with phosphorus oxychloride and a fluoride source (NaF, KF, HF etc) can also be used to functionalise a monomer, polymer or macromolecule. The procedure is modified by two different procedures by Stolzer, C.; Simon, A.; Chem. Ber;, 1960, 93, 1323 and Ford-Moore, A. H.; Lermit, L. J.; Stratford, C.; J. Chem. Soc.; 1953, 1776.

The alcohol (18.00 mmol) was added slowly to a solution of phosphorus oxychloride (2.84 g, 18.00 mmol) in carbon tetrachloride (10 ml) under nitrogen. The addition was accompanied by a slight exotherm. The resulting mixture was stirred at room temperature overnight. The next day, the solvent (and HCl) was removed in vacuo to leave a colourless liquid. A suspension of sodium fluoride (0.76 g, 18.00 mmol) in carbon tetrachloride (10 ml) was warmed to 40° C. To this suspension was added the colourless liquid. The reaction mixture was then refluxed for one hour. The precipitate that had formed was filtered off through celite and the filtrate was concentrated in vacuo to leave a pale brown liquid. The liquid was cooled in ice and to this was added sodium hydroxide solution dropwise until pH=7. The water was removed in vacuo to leave a white solid which was dried in a vacuum desiccator over P₂O₅.

The sodium fluoride could routinely be replaced by any other known fluoride source such as potassium fluoride, hydrogen fluoride and Et₃N.3HF.

### EXAMPLE 6

### Reaction of an alcohol with POCl₃ and triethylamine trihydrofluoride.

Phosphorus oxychloride (4.23 ml, 0.045 mol) in 1,4-dioxane (40 ml) was added dropwise to a solution of the alcohol (0.045 mol) at 30° C. and then stirred overnight at room temperature. The solvent was removed in vacuo and the residue was taken up in dichloromethane (40 ml). To this solution was added dropwise triethylamine trihydrofluoride (4.95 ml, 0.030 mol) and the resulting mixture was stirred at room temperature for 3 days under nitrogen. The reaction mixture was then filtered and the residue was taken up in water (20 ml) and neutralised with NaHCO₃ solution. The water was then removed in vacuo to leave the product.

### EXAMPLE 7

### Reaction of a phosphate with oxalyl chloride and triethylamine trihydrofluoride.

Oxalyl chloride (20 ml of a 2 M solution in DCM, 0.040 mol) was added dropwise to a solution of the phosphate (0.020 mol) in dichloromethane (60 ml) at room temperature under nitrogen. The reaction mixture was stirred overnight at room temperature to ensure complete reaction. The next day the reaction mixture was reduced in vacuo and the residue was taken up in fresh, dry dichloromethane (20 ml). To this solution was added triethylamine trihydrofluoride (2.61 ml, 0.016 mol) dropwise and the reaction mixture was stirred for 3 days under nitrogen at room temperature. Diethyl ether (20 ml) was then added and the solution was filtered. The filtrate was neutralised with NaHCO₃ solution, the two layers were separated and the aqueous extract was reduced in vacuo to leave the product.

### EXAMPLE 8

The following represent macromolecules according to the invention.

i) Poly(ethylene glycol methacrylate fluorophosphate), sodium salt

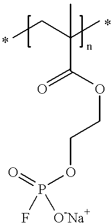

ii) Poly(ethylenimine fluorophosphate), sodium salt

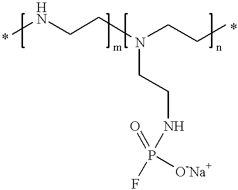

iii) Poly(vinyl alcohol fluorophosphate), sodium salt

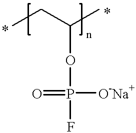

iv) Poly(acrylic acid fluorophosphate), sodium salt

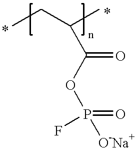

v) Poly(ethylene glycol acrylate fluorophosphate), sodium

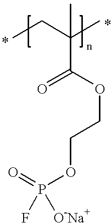

vi) Poly(acrylamide fluorophosphate), sodium salt

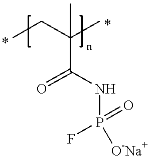

vii) Poly(4-aminostyrene fluorophosphate), sodium salt

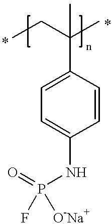

viii) poly(vinylamine fluorophosphate), sodium salt

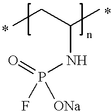

1. A stable oral composition comprising a natural or synthetic macromolecule, said macromolecule comprising a monofluorophosphate moiety covalently bonded thereto.

2. An oral composition according to claim 1, characterised in that the macromolecule comprises a backbone of monomer units terminated by terminal groups and a monofluorophosphate moiety is covalently bonded to a monomer unit.

3. A method of making a macromolecule according to claim 1 or 2 by polymerising a monomer to form a polymer and functionalising the polymer with a monofluorophosphate moiety.

4. A method of making a macromolecule according to claim 1 or 2, characterised by the following steps:
(a) monofluorophosphorylating monomer units; and
(b) (co)polymerising monofluorophosphorylated monomer units,

5. A method of making a macromolecule according to the method of claim 4, characterised in that the polymerisation reaction is a step-addition polymerisation.

6. Use of a macromolecule according to claim 1 or 2 in the manufacture of a medicament for the remineralisation of teeth.
